(19) United States
(12) Patent Application Publication
(10) Pub. No.: US 2003/0055321 A1
(43) Pub. Date: Mar. 20, 2003

(54) System and method for accessing and processing patient data

(76) Inventors: Raymond L. Watrous, Belle Mead, NJ (US); Neal G. Koller, Annapolis, MD (US)

Correspondence Address: Siemens Corporation, Intellectual Property Department, 186 Wood Avenue South, Iselin, NJ, 08830, US

(21) Appl. No.: 10/196,859

(22) Filed: Jul. 16, 2002

Related U.S. Application Data
(60) Provisional application No. 60/305,645, filed on Jul. 16, 2001.

(51) Int. Cl. A61B 5/00

(52) U.S. Cl. 600/300

(57) ABSTRACT

A system and method for accessing and processing auscultatory data and in particular, to a business model that provides on-line services such as storage, retrieval, diagnostic decision support and review of auscultatory data and auscultatory records. A business method is preferably based on a “per use” market model, wherein each “use” of a medical application and related services is treated as a service for which registered and authorized users will make proportional and/or periodic payments based on the requested service(s). Such payments may be in addition to or in lieu of a one-time payment for the purchase of a medical data acquisition device and/or client software, which is associated with the on-line service and which is provided to each registered user for acquiring patient acoustic data. A business model according to the invention may be implemented with any suitable medical diagnostic application that is configured for capturing and processing auscultatory information to provide automated diagnostic evaluation and determination of medical conditions.

### CROSS-REFERENCE TO RELATED APPLICATION

This application claims priority to U.S. Provisional Application No. 60/305,645, filed on Jul. 16, 2001, which is incorporated herein by reference.

### TECHNICAL FIELD

The present invention relates generally to a system and method for accessing and processing auscultatory data and in particular, to a business model that provides services for on-line storage, retrieval, diagnostic decision support and review of auscultatory data and auscultatory records.

### BACKGROUND

In the context of the rapidly increasing cost of health care, the role of the primary care physician (PCP) as a gatekeeper to the resources of the medical system is critical. The challenge in using health care resources in a cost-effective manner is especially acute in the area of evaluation of acoustic data such as cardiac acoustic data and murmurs evaluation.

Indeed, the evaluation of sounds has importance in the diagnosis of a variety of medical conditions affecting cardiac, gastro-intestinal, pulmonary and other anatomical systems. For example, in cardiac diagnosis, a PCP will typically listen to the acoustic sounds of a patient's heart using a stethoscope to identify the primary heart sounds with reference to the sys/diastolic phase of the heart. The PCP will then determine whether there are any abnormal heart sounds present, such as murmurs and/or clicks, by assessing the relative loudness, duration, intensity pattern, spectral quality and time sequence of the heart sounds. The heart sounds are interpreted in terms of the physiological model of the action of the heart muscle, valves and chambers. A hypothesis can be developed about any possible disease states based on the acoustic evidence and knowledge of the patient's medical history. Possible diagnoses are differentiated by varying the placement of the microphone, the patient's posture, or by having the patient execute different maneuvers that accentuate or diminish certain heart sounds. The accumulated evidence is evaluated for the presence of heart disease. It is then decided whether to refer the patient for diagnostic imaging, particularly ultrasound.

The skill of auscultation, however, is inherently difficult, and difficult to acquire because the human auditory system is not well suited to process the acoustic sounds produced by the human heart, under normal or pathological conditions. Indeed, the acoustic sounds generated by the heart are largely below the threshold of human hearing, which worsens with age. Further, auscultation also relies on correctly determining the correspondence of the primary heart sounds with the systolic and diastolic phase of the heart, which is made more difficult when the systolic and diastolic intervals are nearly equal, typically at elevated heart rates. Further, cardiac sounds typically comprise brief, closely spaced events that are difficult to discern by human listeners.

Further, a conventional stethoscope transfers only a small fraction of the acoustic signal at the chest surface to the listener's ears, and filters the cardiac acoustic signal in the process.

Moreover, learning auscultation is difficult because diagnostic instructional manuals rely on subjective descriptions of heart sounds, which require much practice to appreciate. Furthermore, the practice and teaching of the clinical skill of auscultation of the heart has declined among physicians. Studies have demonstrated that physicians can reliably identify only a small number of standard heart sounds and murmurs. Consequently, serious heart murmurs in many patients go undetected by physicians.

The decline in auscultation skills has led to an overreliance on echocardiography, resulting in a large number of unnecessary and expensive diagnostic studies. As a result, reimbursement for echocardiography has recently come under scrutiny by Medicare.

Another problem associated with manual auscultation is that it is extremely difficult, or virtually impossible, for physicians to recall the sounds made by an individual heart from a previous examination. Indeed, for numerous cases, it is advantageous to be able to compare the results of previous auscultatory examinations with the patient's current condition, in order to assess the extent of deterioration in the condition of the heart valves, etc.

Further, the human auditory memory has difficulty in registering distinctively the sounds of the heart, and this difficult is greatly aggravated by listening to the sounds of other hearts, which accumulate in the typical situation where a physician examines many patients each day.

To address the problems associated with manual auscultation, various systems and methods have been developed to provide automated diagnosis of auscultatory data. For instance, intelligent stethoscopes have been proposed for identifying and analyzing heart sounds. Such devices are described, for example, in U.S. Pat. Nos. 5,025,809, 5,010,889, and 5,218,969. One disadvantage associated with systems that provide automated analyses and diagnosis of auscultatory data is that such systems are based on proprietary frameworks and processing platforms, which are not interoperable. Further, because of the different algorithms and models, different auscultation results may be obtained using different systems.

It would be highly advantageous to develop a system for automated auscultation that would provide a standard for auscultation analysis and diagnosis and that would be accessible from any location at any time using any suitable access device. Indeed, it is anticipated that advanced signal and information processing technologies, deployed on inexpensive computational platforms, will be used to process data from multiple noninvasive sensors to provide accurate and intelligible assessments of patient health status. These assessments will be used by physicians and other healthcare providers to make referral decisions and by healthcare consumers to monitor their own health and to participate more intelligently in decisions regarding their own health care.

One such application development environment that would be suitable for such a system/service is the Internet, for example. The Internet and Internet-related services have been growing at a significant rate due to the ubiquity and granularity of the Internet. Indeed, the Internet and the world Wide Web (WWW) are essentially one of the largest repositories and sources of information and services that are available today. Indeed, on-line services are ubiquitous and readily available to persons using suitable equipment to access sites to download information or access desired services. Access devices (or Internet appliances) comprise all computer-based machines/devices that are capable of communicating with other machines/servers on the Internet using open and interoperable communication standards, such as the Internet suite of IP (Internet Protocols) such as HTTP, WAP, etc,, and using suitable web browsers.

Accordingly, it would be desirable and highly advantageous to have a business model for providing on-line storage, retrieval, diagnostic decision support, review and referral of auscultatory patient medical record data.

### SUMMARY OF THE INVENTION

The present invention is directed to a system and method for accessing and processing auscultatory data and in particular, to a business model that provides on-line services such as storage, retrieval, diagnostic decision support and review of auscultatory data and auscultatory records.

In one aspect of the invention, a business method is preferably based on a “per use” market model, wherein each “use” of a medical application and related services is treated as a service for which registered and authorized users will make proportional and/or periodic payments based on the requested service(s). Such payments may be in addition to or in lieu of a one-time payment for the purchase of a medical data acquisition device and/or client software, which is associated with the on-line service and which is provided to each registered user for acquiring patient acoustic data.

In another aspect of the invention, a business model according to the invention may be implemented with any suitable medical diagnostic application that is configured for capturing and processing auscultatory information to provide automated diagnostic evaluation and determination of medical conditions. The application executes on one or more application servers and/or client devices.

In yet another aspect, a business method employs an access control mechanism to prevent a registered user from accessing a medical service if the user has not performed a predetermined on-line function associated with the medical application. The predetermined on-line functions comprise, for example, a training program that allows a registered user to learn how to use the online services and data acquisition device, a post-training program, routine downloading of software updates for the data acquisition device and/or client software, and a calibration process to calibrate the data acquisition device.

In another aspect, a business method provides on-line medical services such as diagnostic evaluation of auscultatory data, access to patient auscultatory records, selection of a referral entity and sending patient auscultatory records to the selected entity, and automated serial comparison of auscultatory records of a patient to track changes in the acoustic features acquired from the patient over a desired period of time.

In yet another aspect, a business method employs an access control mechanism for configuring the data acquisition device to operate upon purchasing a predetermined number of prepaid uses for an on-line medical service and/or configuring the data acquisition to cease operating when the predetermined number of prepaid uses have been exhausted.

These and other aspects, features and advantages of the present invention will become apparent from the following detailed description of preferred embodiments, which is to be read in connection with the accompanying drawings.

### DETAILED DESCRIPTION OF PREFERRED EMBODIMENTS

The present invention is directed to a business model for a medical application that provides, e.g., on-line archival storage and retrieval of medical data, and server-side diagnostic decision support and referral mechanisms. More specifically, the invention is directed to a business model for a medical application that provides on-line diagnostic decision support for auditory evaluation and diagnosis of anatomical features. In a preferred embodiment of the invention, a business model is based on the systems and methods described in U.S. patent application Ser. No. 09/753,162, filed on Jan. 1, 2001, entitled “A System for Processing Audio, Video and Other Data For Medical Diagnosis and Other Applications,” and U.S. patent application Ser. No. 09/670,284, filed on Sep. 25, 2000, entitled “Multi-Modal Cardiac Diagnostic Decision Support System and Method,” which are both commonly assigned and fully incorporated herein by reference.

Although the present invention is preferably based on, and will be explained with reference to, an on-line service for processing cardiac acoustic signals, it is to be understood and appreciated that a business model according to the invention may be implemented with any suitable medical diagnostic application that is configured for capturing and processing auscultatory information to provide automated diagnostic evaluation and determination of medical conditions. For instance, the invention may provide an on-line service for diagnostic support for auditory evaluation of functions such as pulmonary, gastro-intestinal, obstetrical and fetal, vocal and skeletal functions.

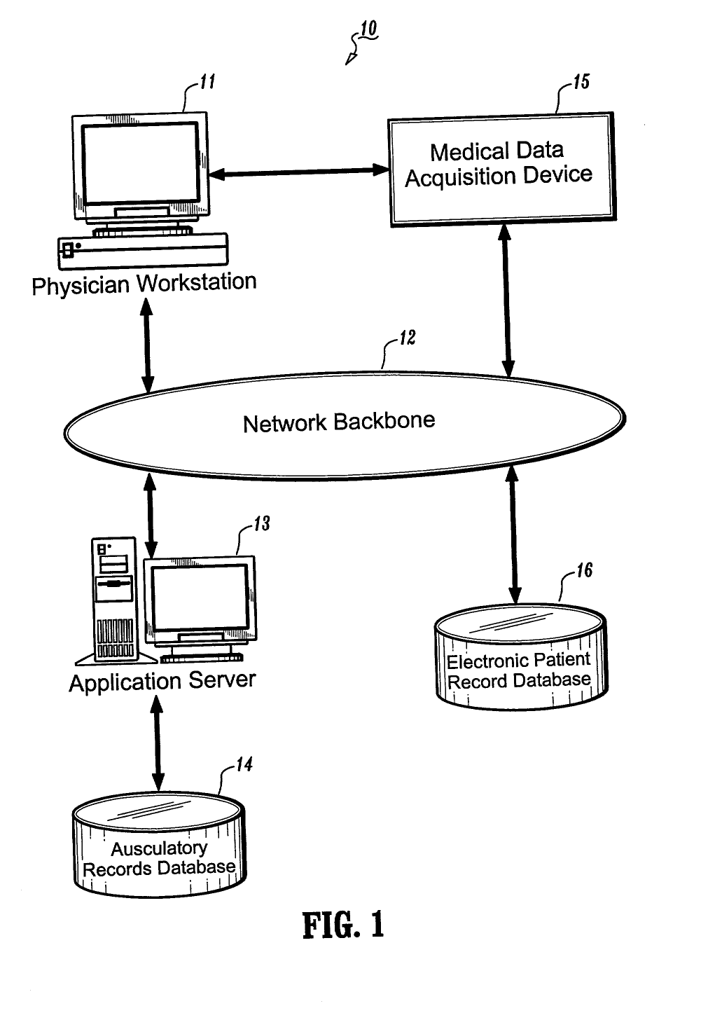

Referring to FIG. 1, a block diagram illustrates a system 10 which implements a business model according to an embodiment of the invention for providing on-line medical services such as archival storage and retrieval of medical data, and server-side diagnostic decision support and referral mechanisms. The system 10 comprises a physician workstation 11, a network 12, a host machine comprising an application server 13, an auscultatory records database 14, a medical data acquisition device 15 and an electronic patient records database 16.

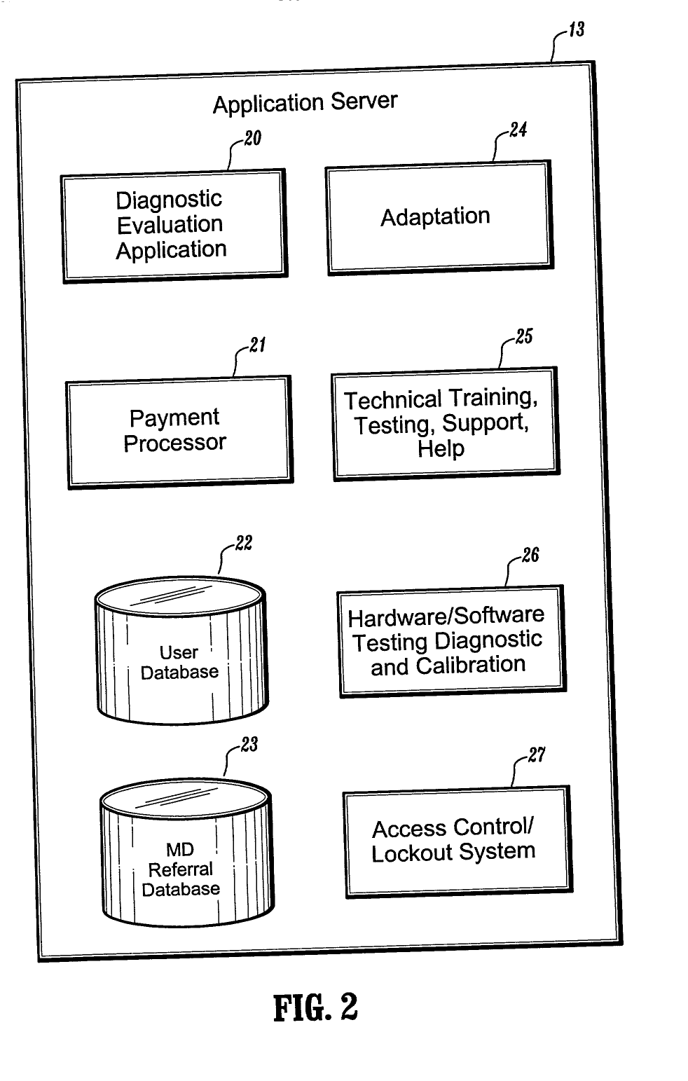

The client workstation 11 comprises appropriate user interface software for accessing and interacting with the application server 13 over the network 12, as well as interacting with the medical data acquisition device 15. In general, the application server 13 comprises an application for providing medical services such as archival storage and retrieval of medical data, and server-side diagnostic decision support and referral mechanisms, as well as other services and functions as described in further detail below with reference to FIG. 2, for example.

In one preferred embodiment, the application server 13 comprises a diagnostic evaluation application (as shown in FIG. 2) that, in general, analyzes cardiac acoustic signals, extracts clinical findings, and generates an estimate of the probability of each of several heart diseases consistent with the clinical finding derived from the cardiac acoustic signals and a patient medical record data retrieved from the database 16, as well as other medical information as discussed below. Based on these analyses, the server 13 provides diagnostic decision support to the PCP in deciding whether to refer the patient for diagnostic test, such as for example, ultrasound or more specifically, echocardiogram.

In one preferred embodiment, the system 10 implements a business model that is preferably based on a “per use” market model, wherein the use of the system 10 is treated as a service for which registered and authorized users will make proportional and/or periodic payments based on the requested services. Such payments are in addition to or in lieu of a one-time payment for the purchase of the medical data acquisition device 15 that comprises an acoustic sensor, signal processing unit and user interface software.

In a preferred embodiment, the medical data acquisition device 15 comprises suitable architecture for capturing and processing acoustic information such as cardiac acoustic signals. The device 15 may comprise any suitable device such as a portable PDA (personal digital assistance) device comprising suitable architecture for capturing and processing signal acquisition capabilities and interfacing with workstation 11 using known interfaces such as Bluetooth. For example, the device 15 may comprise any known electronic stethoscope that can be interfaced with the workstation 11. Further, the device 15 may comprise a handheld sensor for acoustic data acquisition as described in U.S. patent application Ser. No. 09/670,053, filed Sep. 25, 2000 and Ser. No. 09/893,118, filed on Jun. 27, 2001, which are both entitled, “Handheld Sensor For Acoustic Data Acquisition”, and which are commonly assigned and fully incorporated herein by reference.

A PCP uses the data acquisition device 15 to acquire acoustic data representative of an acoustic signal associated with an anatomical function. Depending on the processing capabilities of the device 15, the acquired acoustic data can be preprocessed (e.g., filtered, feature extraction, encryption, compression) using appropriate software running in the device 15 or workstation 11. The acoustic signals that are acquired via device 15 are digitized and securely transmitted to the application server 13 either directly from the device 15 or from the workstation 11.

The patient database 16 comprises a central repository of patient data for one or more patients. The database 16 comprises patient medical information (such as clinical conditions and/or sub condition) and is accessible over the network 12 by the client workstation 11 and application server 13. The patient data in database 16 comprises medical information associated with one or more patients, which can be used by the application server 13 for providing diagnosis of acoustic data. The medical information may include, for example, symptoms, history and results of physical examinations, as well as acoustic data acquired from the patient.

The application server 13 can be accessed to automatically analyze the acquired acoustic data and medical record information to determine physiologically significant features useful in medical diagnosis. The results of such analysis are stored in auscultatory records database 14. As explained below, the accumulation of auscultatory records in database 14 provides various advantages such as providing a central managed repository for easy access to the records and providing a mechanism for continuously adapting/optimizing the diagnostic evaluation algorithms running in application server 13.

It is to be understood that although a preferred embodiment as depicted in FIG. 1 comprises a client-server framework, the system 10 may be implemented using any suitable computing environment framework such as P2P (peer-to-peer) or master/slave, for example. It is to be understood that the network 12 comprises the infrastructure and backbone associated with an Intranet, a LAN (local area network), WAN (wide area network), a P2P network, a global computer network (e.g., Internet), a wireless communications network, PSTN (public switched telephone network) or any combination thereof, utilizing suitable communication protocols. Further, while the illustrative embodiment of FIG. 1 describes a single server machine 13 for providing diagnostic decision and support, those skilled in the art will understand that the functionality of application server 13 may be distributed over the network on different host machines. Those of ordinary skill in the art can readily envision various architectures for implementing a system 10 according to the invention based on the teachings herein and nothing herein shall be construed as a limitation of the scope of the invention.

In one embodiment, the application server 13 comprises the business logic for providing on-line medical services such as archival storage and retrieval of auscultatory medical data, and server-side diagnostic decision support and referral mechanisms, as well as other services and functions as will now be described in detail. FIG. 2 is a high-level diagram illustrating software components of an application server 13 according to an embodiment of the invention. Referring to FIG. 2, the application server 13 comprises a diagnostic evaluation application 20 for processing acoustic data and other medical information to provide diagnostic evaluation of one or more conditions. It is to be understood that depending on the processing capabilities and resources of the client workstation 11 and data acquisition device 15, the business logic (e.g., the diagnostic application 20) of application server 13 may be distributed over the application server 13 (or other servers) and the client 11 and/or device 15. In another embodiment, the application 20 may reside solely on the client 11 and/or device 15. Thus, the architecture of the application server 13 depicted in FIG. 2 is just one embodiment of the invention and should not be construed as providing any limitation of the invention.

It is to be further understood that a business model according to the present invention may be based on any medical application for diagnosis and treatment. As noted above, in one preferred embodiment, the application 20 is based on the multi-modal cardiac diagnostic decision support system and method disclosed in the above-incorporated U.S. patent application Ser. Nos. 09/753,162 and 09/670,284.

Briefly, as described in detail in the above incorporated U.S. patent application Ser. Nos. 09/753,162 and 09/670,284, the cardiac diagnostic decision support system processes cardiac acoustic data (which is captured using the data acquisition device 15), patient medical information (symptoms, history and results of physical examination), and other information. A preamplifier and filter amplifies and filters the acoustic signals to increase the signal to noise ratio (these components may reside, for example, in the data acquisition device 15 or client 11). A wavelet decomposition circuit is employed to analyze the filtered signal using a wavelet decomposition to extract time-frequency information. Neural network feature extractors are trained from labeled examples to identify basic heart sounds, clicks and murmurs. In a preferred embodiment, the neural networks are of the time-delay variety, where the input span, number of layers, unit function, connectivity and initial weight selection are appropriately chosen according to well-known methods. A sequence interpreter interprets or parses the extracted features with reference to a state-transition model of the heart to determine the most probable sequence of cardiac events. The state machine can be probabilistic, such as, for example, a hidden Markov model. A duration and phase measurement circuit computes the average state durations of the sequence model. Event sequences are read off from the state trajectory of the sequence model. A clinical findings extractor extracts clinical findings from the acoustic signal based on the state duration, phase and amplitude information, along with dynamic and positional information provided by the user. Any heart murmurs present are quantified relative to the systolic/diastolic interval, and the murmurs are labeled with reference to the phase as early-, mid-, late- or pan-systolic or diastolic. The murmur intensity is scaled by a psychoacoustic measure and graded I through VI. A decision support circuit combines clinical findings with patient medical data input to produce estimates of the posterior probabilities of the covered cardiac diseases. This involves Bayesian networks and expert systems. Dynamic Bayesian networks are preferably used to model the progress of disease states. A signal output device can be used for displaying (e.g., display of client 11 or device 15) or otherwise presenting (printing) the recorded signals to the user. The results of the analysis of the acoustic signal are output to the user (display, printer) A diagnostic output (display, text) provides the results of the diagnostic decision support analysis to the user.

As noted above, a business model according to one embodiment of the invention treats the use of the medical diagnosis and support system as a service for which proportional and or periodic payments are made for particular uses of the system. Referring again to FIG. 2, the application server 13 further comprises a payment processor 21 which preferably employs known methods for providing on-line credit card payment for purchase of “uses” of the system. It is to be understood that the term “use” may comprise any type of interaction or transaction/service with the system whether it be a complex or simple transaction/service. For example, a “use” may comprise an analysis of acoustic data, accessing auscultatory records from database 14, using a referral service, etc. In this regard, there may be different types of “uses” that require different payment schedules. The manner(s) in which a “use” is defined will vary depending on the business model and types of services offered by the system.

In a preferred embodiment, the “uses” of the system are preferably prepaid by credit card. Depending on the desired business model, access to the system may require a minimum monthly quantity of “uses” (in general, or of one particular type) to be purchased by each authorized user, for example. The application server 13 may be configured for tracking the usage of authorized users and provide incentives and/or discounts to high volume users to encourage increased use.

The application server 13 maintains a user database 22 that maintains information associated with registered users of the system including, for instance, name, address, credit card numbers, phone numbers, user ID numbers, IP address(es), system usage information, public/private key information, etc, and other relevant information.

Further, a referral database 23 is maintained by the application server 13. The database comprises information associated with registered MD/experts/labs. The system comprises an on-line referral mechanism that allows a user (PCP) to select a desired doctor/lab from the database 23 and send the auscultatory record of a given patient in database 14 (FIG. 1) to the selected person for further evaluation or a second opinion. The contact information for one or more registered/authorized medical doctors comprises, e.g., name, address, facsimile number, IP address, or any other means for contacting the doctor.

The application server 13 further provides a plurality of on-line functions to ensure quality control. For example, the application server 13 comprises a module 25 for providing on-line technical training, testing, support and help. In a preferred embodiment, a business model requires each registered user to perform an on-line training program for learning how to use the software and how to properly use the associated data acquisition device 15 (FIG. 1). Further, the business model preferably requires each registered user to perform an on-line post training test. The application server 13 preferably comprises an access control/lockout mechanism 27 that prevents the shipment of the data acquisition device 15 to a registered user until the registered user completes the on-line training and verification of completion of the post-training test is verified.

Furthermore, module 25 periodically requires each registered user to perform on-line training updates and perform routine training tests. In a preferred embodiment, the business model requires the on-line routine training and associated tests to be performed before the user can purchase additional “uses”. Indeed, the access control/lockout system 27 will prevent the user from purchasing additional “uses” until it is verified that the user completed the routine training and testing.

The application server 13 further comprises a hardware/software testing, diagnostic and calibration module 26 to provide on-line software and hardware testing, calibration and troubleshooting services. More specifically, by way of example, in a preferred embodiment, the business model provides on-line routine device updates wherein the user can download software updates that are used for operating/configuring the associated data acquisition device and user interface software. In addition, the system may require the user to periodically perform an on-line device calibration process. The lockout system 27 is preferably configured to prevent the registered user from purchasing additional “uses” until the user downloads the appropriate software updates and/or provides on-line verification of the device calibration. In addition, the module 26 provides on-line services such as automatic diagnostic and notification to a registered user when a malfunction of the device or user software is detected by application server 13. The application server 13 further provides on-line repair service via module 26.

As noted above, the access control/lockout system 27 provides on-line control to ensure that each registered user of the system is adequately trained to use the data acquisition device and diagnostic system and to ensure that the data acquisition device is operating at a desired optimal level of performance. The access control/lockout system 27 preferably uses known methods such as access control codes and/or software configuration files/keys to control access/usage of the system. By way of example, the business model preferably provides access control to ensure a minimum per use purchase. This may be performed by configuring the data acquisition device 15 to function upon download of a predetermined number of “uses”. In addition, the device 15 can be configured to cease functioning when the pre-purchases number of “uses” has been exhausted.

The application server 13 further comprises an adaptation module 24 which preferably uses the auscultatory records to periodically update/optimize the signal processing algorithms/models that are implemented by application 20 to process the acoustic data. Adaptation may be performed using known supervised/unsupervised methods, as is understood by those skilled in the art.

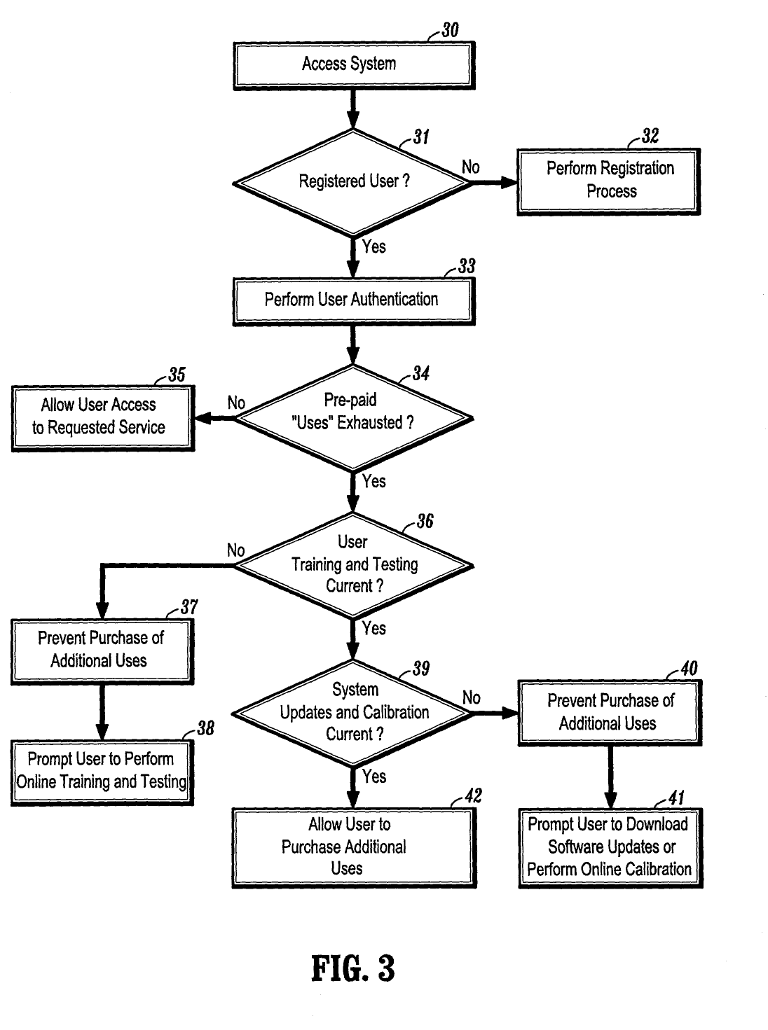

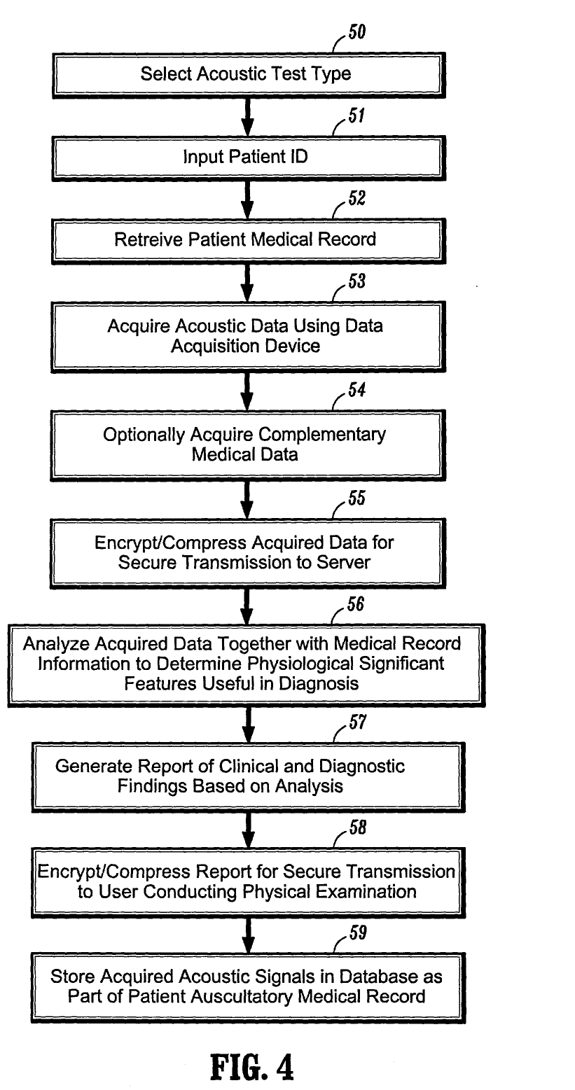

Various embodiments of the invention will now be discussed with reference to the flow diagrams of FIGS. 3 and 4. It is to be understood that the methods described herein in accordance with the present invention are preferably implemented in software comprising program instructions that are tangibly embodied on one or more program storage devices (e.g., magnetic floppy disk, RAM, CD ROM, ROM, etc.), and executable by any device or machine comprising suitable architecture. It is to be further understood that the flow of the process steps may differ depending upon the manner in which the present invention is programmed. Given the teachings herein, one of ordinary skill in the related art will be able to contemplate these and similar implementations or configurations of the present invention.

FIG. 3 is a flow diagram illustrating a business method according to an embodiment of the invention for providing on-line medical services. Initially, a PCP will access the application server using the appropriate access device (step 30). For instance, in one preferred embodiment wherein the application server 13 comprises a web server accessible over the Internet, the access device may comprise a PC (personal computer) running a web browser client application. Alternatively, the access device may comprise a portable “fat client” PDA device that comprises the acoustic data acquisition capabilities. Once the desired auscultatory site is accessed, the user will be prompted to indicate whether the user is a registered user (step 31). If the user is not registered, the user can perform a registration process (step 32). Such process comprises providing personal information, credit card information, setting password/PIN (personal identification number), and any other information that is used to establish a user account.

If the user is registered, the user will be prompted to perform user authentication to verify that the user is an authorized user (step 33). User authentication is performed using any known technique including password, PIN (personal identification number), and/or biometric verification. Once the user is logged in, the system will determine if user's pre-paid “uses” have all been exhausted (step 34). If the user has not exhausted his/her pre-paid “uses” (negative determination in step 34), the user will be allowed to perform a requested service (step 35).

On the other hand, if the user has exhausted all of his/her previously pre-paid “uses” (affirmative determination in step 34), the system will determine the status of the user's training and testing (step 36) and determine whether the user has downloaded the most current software updates and performed a device calibration process (step 39) before the user is permitted to purchase additional uses (step 42). For example, if the user is a first time registrant, and has not performed the initial on-line training program and testing, or if the user has not performed a requisite training update and testing (negative determination in step 36), the user will be prevented from purchasing “uses” (step 37) and the user will be prompted to perform the requisite on-line training and testing (step 38). The user may proceed with the on-line training and testing, in which case the user will thereafter be permitted to purchase additional uses (step 42).

Further, if the user has not downloaded or otherwise acquired the most current software updates for the data acquisition device or the device calibration is not current (negative determination in step 39), the user will be prevented from purchasing “uses” (step 40) and will be prompted to download the appropriate updates and/or perform on-line device calibration (step 41). The user may proceed with a software update or device calibration, in which case the user will thereafter be permitted to purchase additional uses (step 42).

FIG. 4 is a flow diagram illustrating a method for providing diagnostic decision support according to an embodiment of the invention. FIG. 4 is an illustrative method for processing acoustic data according to one embodiment of the invention. It is assumed in FIG. 4 that the user (PCP) has successfully accessed and logged into the desired application server (e.g., auscultatory web site) with the intention of performing a physical examination of a given patient. Initially, using the appropriate access device and user interface modality (e.g., desktop workstation with web browser) the user will select an acoustic test type to be performed (step 50), which identifies the anatomical function being examined such as cardiac, pulmonary, gastro-intestinal, gynecological, skeletal, etc. The user also enters the patient ID (step 51) (or any other patient identifier). The patient ID is used by the application server 13 to retrieve a patient medical record from a central database (step 52). It is to be understood, however, that the patient medical record database may locally reside on the PCP's workstation or local (private) network, in which case the medical record can be transmitted (securely) by the PCP to the application server.

The user will initiate the examination and begin acquiring acoustic data using the data acquisition device (step 53). During the examination, the user will input test type data that identifies the condition of the test including specific location of the organ or body part under examination as well as the condition of the patient and the patient's posture or activity during the test. For example, the data acquisition may be configured for acquiring a cardiac acoustic signal from various sites on surface of the patient's chest, with the patient in various postures (e.g., sitting, standing, reclining, etc.) and under various conditions (e.g., inspiration vs. expiration, static or dynamic, hand-grip, Valsalva maneuver, etc.). For each position of the sensor on the chest surface, the user will input or confirm the sensor position. The sensor position can be inferred with respect to a standard site sequence protocol, guided by the signal interpretation system, confirmed by the user, input by the user using a keyboard, mouse, or position indicator located on the acoustic sensor, or measured with reference to a standard location by a position sensor that is a component of the acoustic data acquisition device.

For both inspiration and expiration and each dynamic maneuver, the user will input or confirm the respiratory phase/maneuver, initiate and terminate signal acquisition and confirm the signal quality (and reacquire the data as needed).

Optionally, the user may acquire complementary medical information associated with the audio data (step 54). The complementary medical information may include video data (representing a sonogram or MRI (magnetic resonance image)), respiratory data, temperature, blood pressure, oximetry or electrical sensory signal or other medically significant signals of value in diagnosis. For example, a respiration signal can be used to provide information about the respiratory activity of the patient for use in the interpretation of the cardiac acoustic signal. The respiratory signal can be obtained, for example, from an impedance respiration sensor or a nasal thermistor sensor. An electrocardiogram (ECG) sensor may optionally be employed to record the surface electrocardiogram to provide a reference signal for synchronizing the recorded acoustic signal.

The acquired acoustic signals (and complementary data) are preferably filtered, digitized, encrypted and compressed for secure transmission (using suitable transport protocols) to the application server (step 55) via the Internet under the control of either the user's PC or a (wireless)-data acquisition device. Preferably, any known compression/encryption scheme may be used that enables accurate and intelligible server-side reconstruction of the acoustic data.

The application server will then analyze the acquired acoustic data, medical record information and complementary medical information for physiologically significant features to determine clinical findings and recommended courses for further actions (step 56). The analysis may include, for example, a comparison of the patient's acoustic data with previously obtained acoustic data retrieved from storage. In addition, the system supports semi-automatic analysis, wherein diagnosis of the acquired acoustic data and other data is performed by a remote operative (e.g., expert physician), wherein the remote expert can enter a clinical opinion and related data via menu screens following his/her analysis of the acoustic data and other data received from the user PCP.

A report of clinical and diagnostic findings is generated based on the automatic/semi-automatic analysis (step 57). Preferably, the report comprises a list of clinical findings together with other findings derived from the medical record. In addition, a conclusion identifying the determined clinical findings as being consistent with one or more medical conditions may be provided. Further, such listed medical conditions may be ranked according to their likelihood of occurrence, or combined with an associated estimate of likelihood of occurrence. The generated information may also include identification of options for further diagnosis or treatment depending on the condition analyzed. The options for further diagnosis or treatment can be ranked according to cost effectiveness, or other criteria such as cost or the informative value of the recommended course of action.

The report is preferably encrypted and compressed for secure transmission to the PCP (step 58).

The acquired signals, extracted features, interpretation and diagnostic results are archived in the auscultatory records database for future reference as part of the patient's auscultatory medical record (step 59). Such records can be made available electronically upon user request for data transfer, or for generation of printed reports.

There are many advantages associated with a business model according to the invention for providing for on-line storage, retrieval, diagnostic decision support, review and referral of patient medical record data. To begin, the distribution and implementation of the system over a public network such as the Internet using open, standard, and interoperable communication protocols and access modalities/devices affords global and ubiquitous access to the clinical information and services from any location at any time, using any suitable access device (PC, PDA, mobile telephone, etc.) Indeed, a patient traveling anywhere in the world can have access to his/her auscultatory records.

Further, the accumulation of a large centralized database of auscultatory records provides numerous advantages. For instance, the various acoustic signals (which are acquired from numerous patients) in the auscultatory database can be further utilized to adaptively improve the performance of the signal processing algorithms that derive clinical information from the signals. This adaptive improvement can be unsupervised or supervised; in the latter case, feedback and confirmation from expert clinical sources is required. For instance, previously generated analysis of clinical findings and diagnosed conditions (automatic or semiautomatic) can be compared with corresponding acquired data and/or data such as independently verified findings that are subsequently acquired and entered into the database. The system can use the database and independent finding in auditing previously generated analyses and in refining decision boundaries that identify different medical conditions. This adaptation enables improvement and evolution of the analytical process to a significantly high state of accuracy. In addition, the adaptation increases the system value over time (which provides a competitive business advantage) and provides a mechanism for continuously adapting the system in accordance with new developments in diagnostic knowledge and test procedures. Such adaptation advantageously enables the system to be the basis of a standard of performance to license to competitors.

Another advantage associated with the accumulation of the patient database is that it allows centralized access to important patient information. Indeed, as noted above, acoustic signals are archived as part of a patient auscultatory medical record. From this archive, the digital signals can be recalled, viewed and replayed for the physician to compare with signals recorded at other times and in other places. The accumulation of the auscultatory records in a central repository allows serial studies to compare past audio with current audio of same patient to detect changes. In one embodiment, the serial study can be performed manually, whereby the physician can perform manual comparisons of a series of auscultatory records by, e.g., listening to various acoustic files, etc.

In another embodiment, a business model-can provide a medical service that offers automatic serial comparison studies for a corresponding fee. Indeed, such comparison can be performed by an appropriate application (running on server and/or client) that tracks changes in the acoustic features acquired from a patient over a desired period of time. For instance, such an application would be able to track and identify if an identified murmur is becoming louder over time, if an identified murmur is lasting for a longer period of time, if a new murmur is occurring in the systolic phase, etc.

Another advantage associated with a centralized managed auscultatory database is that the auscultatory records can be forwarded via the Internet to a specialist in the interpretation of cardiac acoustic signals for review, wherein the specialist can view the acoustic signal graphically and listen to it aurally, and report clinical and diagnostic findings. This report can be immediately transmitted to the physician conducting the physical examination. For example, the auscultatory records can also be forwarded via the Internet to a specialist in noninvasive imaging of the heart using ultrasound, which is a typical imaging modality for evaluating congenital heart disease and valvular heart disease first detected by auscultation.

Although the archival properties can be accomplished locally, the MD must manage data and system, which can be costly and inconvenient. A centralized auscultatory database that is accessible over the Internet provides a less burdensome solution for data base management. Further, a centralized database enables easy access by multiple physicians selected by the patient. For instance, as discussed above, the system provides an on-line referral service whereby the PCP can obtain a second opinion/further analysis of the data. The second opinion or referral can be done over Internet by accessing the central auscultatory database, wherein the auscultatory records can be sent to an expert MD for explanation/second opinion. The MD requesting the second opinion or referral can immediately receive further directions from the MD to obtain more data to clarify unclear or questioned result (e.g., ask patient to squat or stand to repeat procedure).

Although the illustrative embodiments have been described herein with reference to the accompanying drawings, it is to be understood that the invention described herein is not limited to those precise embodiments, and that various other changes and modifications may be affected therein by one skilled in the art without-departing from the scope or spirit of the invention. All such changes and modifications are intended to be included within the scope of the invention as defined by the appended claims.

What is claimed is:

1. A method for providing on-line medical services associated with a network accessible medical application, the method comprising the steps of:
receiving a user request for an on-line medical service;
determining if the user has at least one prepaid use available for the requested service;
allowing access to the requested service, if there is at least one prepaid use available for the requested service; and
if there are no available prepaid uses, preventing the user from purchasing a use if the user has not performed a predetermined on-line function associated with the medical application.

2. The method of claim 1, wherein the predetermined on-line function comprises a training program.

3. The method of claim 2, wherein the predetermined on-line function comprises a post training test.

4. The method of claim 1, wherein the predetermined on-line function comprises downloading a software update for a data acquisition device associated with the medical application.

5. The method of claim 1, wherein the predetermined on-line function comprises performing a calibration process to calibrate a data acquisition device associated with the medical application.

6. The method of claim 1, wherein the on-line medical service comprises diagnostic evaluation of auscultatory data.

7. The method of claim 1, wherein the on-line medical service comprises accessing an auscultatory record associated with a patient.

8. The method of claim 1, wherein the on-line medical service comprises selecting a referral entity and sending an auscultatory record associated with a patient to the selected entity.

9. The method of claim 1, further comprising the step of purchasing a predetermined number of prepaid uses for an on-line medical service.

10. The method of claim 1, further comprising the step of downloading a predetermined number of prepaid uses to enable operation of a data acquisition device associated with the medical application.

11. The method of claim 10, further comprising the step of preventing operation of the data acquisition device when the predetermined number of prepaid uses have been exhausted.

12. A system for providing on-line medical services associated with a network accessible medical application, the system comprising:
a data acquisition device for acquiring acoustic data representative of an acoustic signal associated with an anatomical function;
an application server for providing a plurality of on-line medical services, the on-line medical services comprising a service for automatically analyzing acoustic data, which is acquired with the data acquisition device, to determine physiologically significant features useful in medical diagnosis; and
a auscultatory records database, accessible by the application server, for storing auscultatory records comprising the acquired acoustic data of one or more patients,
wherein the application server comprises an access control mechanism for preventing a user from accessing a medical service if the user has not performed a predetermined on-line function associated with the medical application.

13. The system of claim 12, wherein the predetermined on-line function comprises a training program.

14. The system of claim 13, wherein the predetermined on-line function comprises a post training test.

15. The system of claim 12, wherein the predetermined on-line function comprises downloading a software update for the data acquisition device.

16. The system of claim 12, wherein the predetermined on-line function comprises performing a calibration process to calibrate the data acquisition device.

17. The system of claim 12, wherein the on-line medical services comprise a service for generating a diagnostic report of one or more medical conditions based on an evaluation of auscultatory data.

18. The system of claim 12, wherein the on-line medical services comprise a service for accessing an auscultatory record associated with a patient.

19. The system of claim 12, wherein the on-line medical services comprise a service for selecting a referral entity and sending an auscultatory record associated with a patient to the selected entity.

20. The system of claim 12, wherein the on-line medical services comprise a service for automated serial comparison of auscultatory records of a patient that tracks changes in the acoustic features acquired from the patient over a desired period of time.

21. The system of claim 12, wherein the application server further comprises a payment mechanism for purchasing a predetermined number of prepaid uses for an on-line medical service.

22. The system of claim 12, wherein the application server comprises an access control mechanism for configuring the data acquisition device to operate upon purchasing a predetermined number of prepaid uses for an on-line medical service.

23. The system of claim 22, wherein the access control mechanism configures the data acquisition to cease operating when the predetermined number of prepaid uses have been exhausted.
